(19) United States
(12) Patent Application Publication
(10) Pub. No.: US 2021/0335491 A1
(43) Pub. Date: Oct. 28, 2021

(54) PREDICTIVE ADAPTIVE INTELLIGENT DIAGNOSTICS AND TREATMENT

(71) Applicant: AUGMENTED MEDICAL INTELLIGENCE INC., Golden, CO (US)

(72) Inventors: Scot Morris, Golden, CO (US); Patrick O'Fallon, Golden, CO (US)

(21) Appl. No.: 17/238,435

(22) Filed: Apr. 23, 2021

Related U.S. Application Data
(60) Provisional application No. 63/015,253, filed on Apr. 24, 2020.

(51) Int. Cl. G16H 50/20; G16H 50/50; G16H 10/60; G06T 7/00; G06N 3/08

(52) U.S. Cl. CPC G16H 50/20; G16H 50/50; G06N 3/08; G06T 7/0012; G16H 10/60

(57) ABSTRACT

The present disclosure provides a diagnostic and treatment system for communicating with a device to obtain diagnostic input, generating one or more diagnostic evaluations based on the diagnostic input, and generating a subset of differential diagnoses based on the answers to the diagnostic questions and results corresponding to the one or more diagnostic evaluations. The system also receives a selected diagnosis from the subset of differential diagnoses and generates a subset of treatment options based on the selected diagnosis.

### CROSS-REFERENCE TO RELATED APPLICATIONS

This application claims priority to Provisional Application No. 63/015,253 entitled “Predictive Adaptive Intelligent Diagnostics and Treatment,” filed on Apr. 24, 2020, which is incorporated by reference herein in its entirety, for any purpose.

### BACKGROUND

Conventionally, patient diagnosis is based on a doctor's assessment of the patient's symptoms, such that a patient may have more accurate diagnosis based on the expertise and history of the doctor and staff. This leads to many patients being inaccurately or incompletely diagnosed, and many unnecessary tests, which can cause complications as issues and diseases either go untreated or are treated incorrectly. In some instances, patients are forced to travel to multiple different doctors in order to finally receive an accurate diagnosis, which can be time intensive, expensive, and waste valuable time in treating the disease, resulting in worse outcomes for the patient.

Further, doctors and medical tracking systems do not include and often are unable to determine feedback from other physicians, doctors, patients, that track results of treatments, leading to further inaccurate diagnoses and treatment plans.

### SUMMARY

Example methods are described herein. An example method includes communicating with a user device to obtain diagnostic input, generating one or more diagnostic evaluations based on the diagnostic input, generating a subset of differential diagnoses based on the diagnostic input and results corresponding to the one or more diagnostic evaluations, where the diagnostic input and the results corresponding to the one or more diagnostic evaluations are provided to a model to generate the subset of differential diagnoses, receiving a selected diagnosis from the subset of differential diagnoses, generating a subset of treatment options based on the selected diagnosis, and receiving a selected treatment of the subset of selected treatment options.

Example systems are described herein. An example system includes a communications interface configured to communicate with a user device to receive diagnostic input from the user device, a diagnostic model configured to generate a subset of differential diagnoses based on the diagnostic input, and a treatment model configured to generate a subset of treatment options based on a selected diagnosis selected from the subset of differential diagnoses, where the diagnostic model and the treatment model are configured to update based on a treatment outcome for a selected treatment option from the subset of treatment options.

Example computer-readable media are described herein. An example computer-readable media is encoded with instructions for implementing a system, where the instructions include instructions for communicating with a patient device to obtain diagnostic input, generating one or more diagnostic tests based on the diagnostic input, generating a subset of differential diagnoses based on the diagnostic input and results corresponding to the one or more diagnostic evaluations, wherein the diagnostic input and the results corresponding to the one or more diagnostic tests are provided to a model to generate the subset of differential diagnoses, receiving a selected diagnosis from the subset of differential diagnoses, and generating a subset of treatment options based on the selected diagnosis.

Additional embodiments and features are set forth in part in the description that follows, and will become apparent to those skilled in the art upon examination of the specification or may be learned by the practice of the disclosed subject matter. A further understanding of the nature and advantages of the present disclosure may be realized by reference to the remaining portions of the specification and the drawings, which forms a part of this disclosure. One of skill in the art will understand that each of the various aspects and features of the disclosure may advantageously be used separately in some instances, or in combination with other aspects and features of the disclosure in other instances.

### BRIEF DESCRIPTION OF THE DRAWINGS

The description will be more fully understood with reference to the following figures in which components are not drawn to scale, which are presented as various examples of the present disclosure and should not be construed as a complete recitation of the scope of the disclosure, characterized in that:

FIG. 1 is a schematic diagram of an example use of a diagnosis and treatment system;

FIG. 2 is a schematic diagram of an example diagnosis and treatment system;

FIG. 3 is a schematic diagram of an example computer system for implementing various embodiments in the examples described herein.

FIG. 4 is a flow diagram for use of an example diagnosis and treatment system;

FIG. 5 is a flow diagram for use of an example diagnosis and treatment system;

FIG. 6 is a flow diagram for use of an example diagnosis and treatment system.

### DETAILED DESCRIPTION

According to the present disclosure, a diagnostic and treatment system generates a subset of differential diagnoses for a patient based on answers to diagnostic questions presented by the patient. The diagnostic and treatment system may further generate and/or update treatment options after receipt of a selected diagnosis from the subset of differential diagnoses, as well as after tracking a patient's progress and conformity with a prescribed treatment plan. Further, the diagnostic and treatment system may adapt over time through use of the system to diagnose and treat patients. For example, the diagnostic and treatment system may incorporate machine learning, artificial intelligence, or other algorithms to incorporate feedback and improve the system over time.

Diagnosis and treatment of a patient generally includes taking a patient history and making decisions about a patient's diagnosis or treatment based on the patient's history, current symptoms, and a physical review of symptoms. Generally, patient history is taken orally, which may result in missed information or inefficient use of time. Further, accurate patient history may be dependent on the knowledge, experience, and communication skills of medical staff. Diagnostic models, including various software applications may be used to diagnose patients. However, many diagnostic models do not receive feedback about, for example, treatments, patient outcomes, additional diagnoses, or new science or treatment protocols and, accordingly, may not produce accurate results. Further, many providers may be reluctant to use diagnostic models which present a diagnosis without additional provider input.

The diagnostic and treatment system described herein combines patient history information and current symptoms with a knowledge base of information about symptoms, diagnoses, and treatments. The diagnostic and treatment system also provides multiple diagnosis and treatment options such that a provider can use their experience, judgment, and physical examination of the patient to choose a diagnosis and a treatment for the diagnosis. The diagnosis and treatment system may also track patient compliance with the treatment plan and update its knowledge base based on patient treatment outcomes.

The diagnosis and treatment system described herein may provide output and receive diagnostic input in several spoken human languages and may be configured to extract or obtain relevant medical information from, for example, free-form text input. For example, the diagnosis and treatment system may operate using its own encoding scheme encoding information including, for example, information type (e.g., diagnostic input, diagnosis, treatment) along with additional medical information. The encoding may be a tag or other identifier associated with the information itself. Information and data stored within the system may be converted from human language to the common system language such that the system can operate in multiple spoken languages while efficiently analyzing data stored in the system.

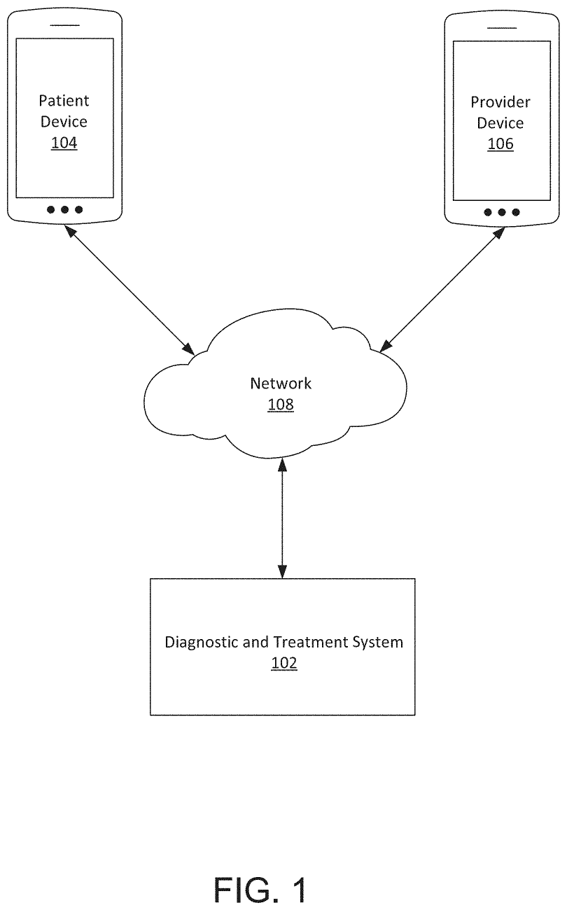

The diagnostic and treatment system 102 shown in FIG. 1 communicates with a patient device 104 and a provider device 106 to diagnose a patient and/or generate treatment options for the patient based on a selected diagnosis. Generally, the diagnostic and treatment system 102 includes a diagnostic model that communicates with the patient device 104 to obtain diagnostic input about the patient. For example, a diagnostic model may generate and present background questions to the patient, where the answers are inputs to the model. In some implementations, background questions may be presented to another user device, such as the provider device 106, and relayed to the patient. Further, the provider may answer additional questions or provide additional input into the system, such as patient test results, patient history, or the like. Other types of diagnostic input, such as data detected from a device, e.g., a patient wearable or implanted biometric devices, may utilized as diagnostic input. For example, a patient may wear a biometric or other sensing device that collects data regarding certain biological or other characteristics of the patient, which can then be utilized by the diagnostic model.

Utilizing the diagnostic input, the diagnostic and treatment system 102 may determine additional diagnostic testing, which can be provided to the provider. The provider may then order the suggested testing or provide previous test results for the suggested diagnostic tests. In other words, based on the initial diagnostic input, the diagnostic and treatment system 102 may determine that additional or supplemental diagnostic input could enhance the diagnosis. Based on this determination, the supplemental input can be collected, e.g., via additional testing, additional patient questions, or the like. Based on the received information, including diagnostic input and test results associated with the suggested diagnostic testing, the diagnostic model determines and presents a subset of differential diagnoses to the provider device 106. The provider may then select a diagnosis from the subset of differential diagnoses generated by the diagnostic and treatment system 102 using the provider's own medical knowledge and clinical decision making, experience, knowledge of the patient, etc. A treatment model of the diagnostic and treatment system 102 may present treatment options to the provider device 106 based on the provider's selected diagnosis.

Over time, the diagnostic model may adjust its algorithms or other models by analyzing diagnoses most commonly selected by providers in conjunction with options determined by the system. That is, the diagnostic model may utilize feedback to update predications and recommendations. Accordingly the diagnostic and treatment system 102 may benefit from the medical knowledge and experience of providers using the system. For example, in some implementations, the diagnostic model may be implemented using an expert system, where, during use of the system, an inference engine interacts with a knowledge base to update the knowledge base. Over time, the knowledge base becomes more robust as it incorporates additional information generated through the diagnosis and treatment of patients using the diagnostic and treatment system 102. The diagnostic and treatment system 102 may, in some implementations, receive additional information, such as patient outcomes, to refine the diagnostic model, the treatment model, or both.

In an exemplary use of the diagnostic and treatment system 102, a patient uses the patient device 104 or another user device to answer a set of diagnostic questions regarding the patient's condition, symptoms, and/or health or relevant history. For example, the patient may be experiencing eye itching and redness. The diagnostic and treatment system 102 may present the patient questions about duration of symptoms, severity of symptoms, whether activities make the symptoms worse, or any other information that may help to identify the cause of the patient's symptoms (which will vary based on the symptoms). In some implementations, the patient may use the patient device 104 to take a picture of the affected areas and the diagnostic and treatment system may further use the image and/or other diagnostic input alongside or in place of diagnostic questions to generate differential diagnoses.. Where answers to diagnostic questions are received from the patient device, the history and symptom review can take place before an appointment with the provider, possible reducing wait times, directing a patient to an appropriate provider, and allowing for telehealth options.

The diagnostic input, such as patient answers to the diagnostic questions, may be used by the diagnostic and treatment system 102 to generate a list of suggested diagnostic testing or evaluations to present to a provider. Tests or evaluations included in the list of suggested diagnostic evaluations may be likely to help exclude or include some diagnoses. The provider may order some or all of the suggested diagnostic tests and may provide the system 102 with the patient's previous results corresponding to some or all of the suggested diagnostic tests. The diagnostic input and the results corresponding to the suggested diagnostic tests are used by the diagnostic and treatment system 102 to generate a list of differential diagnoses for the patient.

The list of differential diagnoses generated by the diagnostic and treatment system 102 is generally presented to the provider through the provider device 106. The diagnostic and treatment system 102 may generate the list of differential diagnoses. The provider may then select a diagnosis from one or more differential diagnoses based on the provider's impressions of the patient (such as a physical examination or additional tests). In some implementations, the diagnostic and treatment system 102 may suggest additional specific tests or evaluations to the provider to eliminate or confirm one or more of the differential diagnoses. In these implementations, the diagnostic and treatment system 102 may also present a standard subset of possible results of the diagnostic test. Once the provider has selected a diagnosis, the diagnosis may be used by the treatment model of the diagnosis and treatment system to generate treatment options for the patient.

The provider, using the provider device 106, may then select a treatment option from the treatment options generated by the diagnostic and treatment system 102. In some implementations, the diagnostic and treatment system 102 may provide additional information to the patient regarding the diagnosis, the treatment, or both, and may supplement and/or track the patient's compliance with the selected treatment (e.g., by reminding the patient each day to use prescribed medication and prompting the patient to check a box after use of the prescribed medication).

The diagnostic and treatment system 102 may collect additional information about the patient during follow-up visits (e.g., treatment outcome, a different diagnosis, or a different treatment options) and use the additional information, along with patient compliance information, to update the models of the diagnostic and treatment system 102. This allows the diagnostic and treatment system 102 to become more accurate over time in diagnosing patients and in providing recommended treatments for different diagnoses. The diagnostic and treatment system 102 may also be updated using published articles, studies, and other medical literature, allowing providers to adopt new treatments quickly. Accordingly, over time, providers using the diagnostic and treatment system 102 are able to benefit from the input and expertise of other providers who have used the diagnostic and treatment system 102.

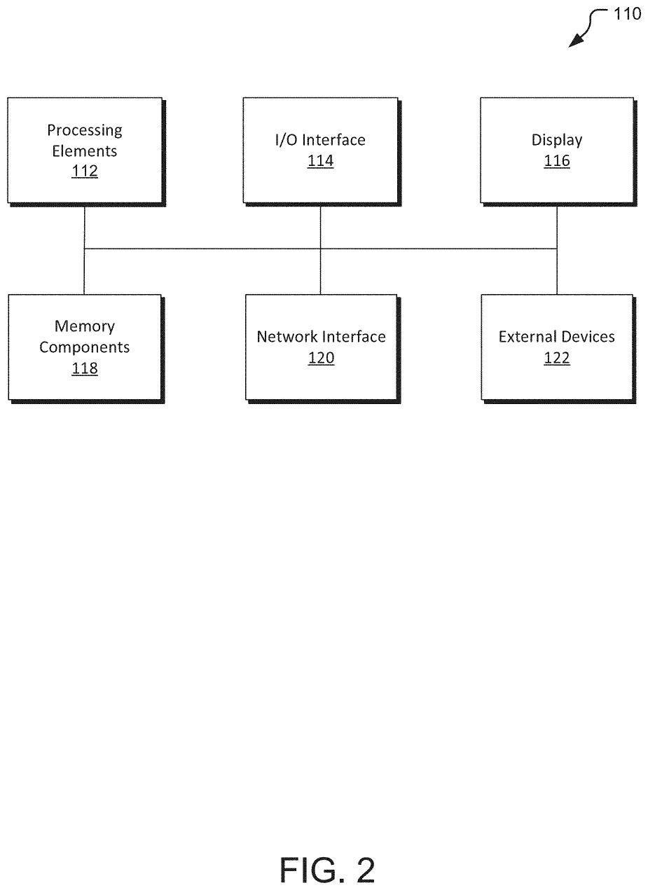

FIG. 2 is a schematic diagram of an example computer system for implementing various embodiments in the examples described herein. A computer system 110 may be used to implement the patient device 104 or the provider device 106 (in FIG. 1) or integrated into one or more components of the diagnostic and treatment system 102. The computer system 110 may include one or more processing elements 112, an input/output interface 114, a display 116, one or more memory components 118, a network interface 120, and one or more external devices 122. Each of the various components may be in communication with one another through one or more buses, communication networks, such as wired or wireless networks.

The processing element 112 may be any type of electronic device capable of processing, receiving, and/or transmitting instructions. For example, the processing element 112 may be a central processing unit, graphical processing unit, microprocessor, processor, or microcontroller. Additionally, it should be noted that some components of the computer system 110 may be controlled by a first processor and other components may be controlled by a second processor, where the first and second processors may or may not be in communication with each other (e.g., through a wired or wireless network). The processing elements 112 may also, in various implementations, include various processing resources which may be distributed across various physical locations and pieces of hardware. The processing element 112 may also include advanced computational elements or methods, such as quantum computation.

The memory components 118 are used by the computer system 110 to store instructions for the processing elements 112, as well as store data, such as the patient data and the like. The memory components 118 may be, for example, magneto-optical storage, read-only memory, random access memory, erasable programmable memory, flash memory, or a combination of one or more types of memory components. The memory components 118 may also include advanced memory and storage, such as crystal storage or multi-dimensional optical storage systems. The memory components 118 may also include distributed memory components which may, in some implementations, be aggregated in a storage pool, virtual file system, or the like.

The display 116 provides visual feedback to a user, such as a display of the patient device 104 (FIG. 1). Optionally, the display 116 may act as an input element to enable a user to control, manipulate, and calibrate various components of the diagnostic and treatment system 102 (FIG. 1), patient device 104, provider device 106, or other computing devices as described in the present disclosure. The display 116 may be a liquid crystal display, plasma display, organic light-emitting diode display, and/or other suitable display. The display may further be a virtual or augmented reality display, such as a heads up display or wearable display. In embodiments where the display 116 is used as an input, the display may include one or more touch or input sensors, such as capacitive touch sensors, a resistive grid, or the like.

The I/O interface 114 allows a user to enter data into the computer system 110, as well as provides an input/output for the computer system 110 to communicate with other devices or services (e.g., patient device 104, physician device 106 and/or other components in FIG. 1). The I/O interface 114 can include one or more input buttons, touch pads, and so on. The I/O interface may also include sensors such as motion sensors and/or cameras to interpret input gestures, microphones, and the like.

The network interface 120 provides communication to and from the computer system 110 to other devices. For example, the network interface 120 allows the diagnostic and treatment system 102 to communicate with the patient device 104 and the physician device 106 (FIG. 1) through a communication network. The network interface 120 includes one or more communication protocols, such as, but not limited to WiFi, Ethernet, Bluetooth, cellular data, and so on. The network interface 120 may also include one or more hardwired components, such as a Universal Serial Bus (USB) cable, or the like. The configuration of the network interface 120 depends on the types of communication desired and may be modified to communicate via WiFi, Bluetooth, and so on.

The external devices 122 are one or more devices that can be used to provide various inputs to the computing system 110, e.g., mouse, microphone, keyboard, trackpad, or the like. The external devices 122 may be local or remote and may vary as desired. In some examples, the external devices 122 may also include one or more additional sensors.

While examples described herein may focus on a centralized computer system 110, the computer system 110 may also be a distributed system, cloud system, server, or the like. Further, a computer system 110 may include components from several different locations. For example, the computer system 110 may utilize local processing resources while utilizing cloud storage locations. The computer system 110 may utilize various computing and data structures such as virtual machines, containers, blockchain, and the like.

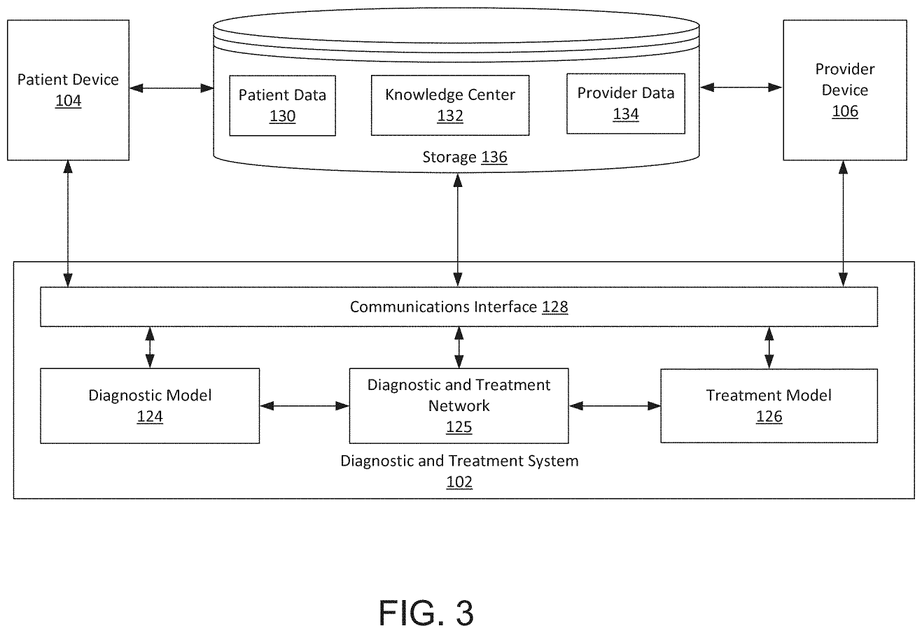

FIG. 3 shows a schematic diagram of the example diagnostic and treatment system 102. The diagnostic and treatment system 102 includes a diagnostic model 124, a diagnostic and treatment network 125, and a treatment model 126. The diagnostic and treatment system 102 is configured to communicate with the patient device 104, storage 136, and the provider device 106 via a communications interface 128. The patient device 104 and the provider device 106 may also communicate with the storage 136. The storage 136 may include patient data 130, a knowledge center 132, and provider data 134.

The diagnostic and treatment system 102 may be implemented using one or more computing devices, such as the computing system 110. For example, in one implementation, the diagnostic and treatment system 102 is implemented at a server, where the diagnostic model 124 is implemented using one processor and memory resources at the server and the treatment model 126 is implemented using another processor and memory resources at the server. The diagnostic and treatment network 125 may further be stored or accessed using processing and memory resources at another physical machine or location. In other implementations, the diagnostic model 124 and the treatment model 126 may be implemented using the same processor or physical machine. The diagnostic and treatment system 102 may also be implemented within a cloud computing environment, using a distributed computing system, using multiple virtual machines, or using other types of computational systems.

The diagnostic and treatment system 102 generally includes a communications interface 128 that receives communications from, for example, the patient device 104 and the provider device 106 and parse those communications to provide input to, for example, the diagnostic model 124 and the treatment model 126. The communications interface 128 may, in some implementations, include multiple interfaces or multiple types of logic to receive communications through different types of networks (e.g., wireless internet, cellular data, etc.). The communications interface 128 also sends communications to devices using the diagnostic and treatment system 102. For example, the communications interface 128 may send diagnostic questions to the patient device 104 and send differential diagnoses and treatment options to the provider device 106. The communications interface 128 may further provide communication to additional devices and systems which may provide diagnostic input, receive information from the diagnostic and treatment system 102, and the like. For example, the communications interface 128 may receive data from external devices, such as patient wearables (e.g., biometric devices), electronic health record (EHR) systems, and other data sources. The communications interface 128 may also manage requests by the diagnostic and treatment system 102, or by devices using the diagnostic and treatment system 102 to access remote or local storage. The communications interface 128 may employ various security protocols such as encryption, multi-factor authentication, or firewalls to control communications with and access to the diagnostic and treatment system 102.

The patient device 104 and the provider device 106 may include user devices such as desktop personal computers, mobile phones, laptops, tablets, wearable computers, implanted or wearable devices or other computing devices capable of connecting to the network 108 and communicating with the diagnostic and treatment system 102, such as described herein. The patient device 104 and the provider device 106 may further include various user interfaces and peripheral components that allow a user to receive information from and provide information to the patient device 104 and the provider device 106. For example, a keypad, touch screen, display, camera, microphone, speakers, or other hardware components may allow users to interact with the patient device 104 and the provider device 106. For example, a camera in the patient device 104 may allow a patient to provide an image to the diagnostic and treatment system 102 as diagnostic input. Further, the patient device 104 may be a wearable device with various sensors (e.g., heart rate sensors, accelerometers, etc.) which may provide input data to the diagnostic and treatment system 102. In another example, speakers may be used in lieu of or in addition to a display to provide diagnostic questions to a patient. In some implementations, other user devices may also communicate with the diagnostic and treatment system 102.

The patient device 104 and the provider device 106 may, in some implementations, be the same user device. For example, a provider may present a device to the patient to answer patient questions. The provider may then use the same device to select a diagnosis and treatment options. In some implementations, additional devices may provide input to the diagnostic and treatment system 102. For example, diagnostic questions may be presented to a caregiver through an additional computing device or, in some implementations, the patient device 104 may be eliminated.

Multiple patient devices and provider devices may simultaneously access and use the diagnostic and treatment system 102. In these implementations, the patient devices and provider devices may be associated with user identifiers, passwords, or other secure identifiers to determine which parts of the diagnostic and treatment system 102 may be accessed by a particular device. For example, the patient device 104 may be associated with a particular patient and may be unable to access the treatment model 126 to generate treatment options based on a selected diagnosis. Further the identifiers of the patient device 104 and the provider device 106 may be linked to allow communication between the patient device 104 and the provider device 106 or to allow the patient device 104 and the provider device 106 to share certain information.

The diagnostic model 124 generally receives as input responses to patient questions and generates a set of differential diagnoses based on the responses. In some implementations, the diagnostic model 124 may also guide the process of presenting patient questions (e.g., by determining a next question to present based on the answer to a previous question).The diagnostic model 124 may be implemented using various algorithms, machine learning models, or combinations of both. For example, in one implementation, the diagnostic model 124 may be a classifier trained using expertly seeded data. In other implementations, an initial algorithm, created with input from experts (e.g., physicians or medical providers) may be continually updated with data generated from treating patients using the diagnostic and treatment system 102.

The diagnostic model 124 may also be implemented using an expert system including an inference engine trained to interact with and update a knowledge base created within the diagnostic and treatment system 102, such as the diagnostic and treatment network 125. In some implementations, the diagnostic model 124 may incorporate additional modules, models, or software to process input data. For example, an imaging processing model may receive and process an image of a patient's eye, providing additional input to the diagnostic model 124 to generate differential diagnoses. For example, the diagnostic model 124 could include a computer vision model developed to identify conditions based on images, such as ophthalmological or dermatological conditions, e.g., the computer vision model could use color and morphology detected in an image to assess conditions. An image processing model may also, in some implementations, receive and analyze resultant images from, for example, magnetic resonance imaging (MRI), a computed tomography (CT) scan, ultrasound, or other various diagnostic imaging methods. Such images could be analyzed using computer vision or other types of image analysis.

The diagnostic model 124 may also include models to generate suggestions for diagnostic testing based on diagnostic input. Such models may suggest testing that is likely to narrow down possible diagnoses to a subset of differential diagnoses. In some examples, diagnostic testing models may further suggest repeating previously performed tests to, for example, monitor changes in values or to obtain higher quality results, such as clearer or higher resolution imaging.

The treatment model 126 generally receives as input a selected diagnosis from the set of differential diagnoses and generates treatment options based on the selected diagnosis. The treatment model 126 may be implemented using various algorithms, machine learning models, or combinations of both. For example, in one implementation, the treatment model 126 may use an algorithm to provide set treatment options for a particular diagnosis. In other implementations, the treatment model 126 may use clustering to select treatment options with a higher likelihood of success for a particular condition based on, for example, treatment data received from a provider or patient indicating whether a treatment was successful. In yet another implementation, the treatment model 126 may be implemented using an expert system, which may include a knowledge base and an inference engine. The treatment model 126 may also receive information from other sources, such as public or subscription databases, medical journals, or other sources of clinical information. The treatment model 126 may present treatment options with an indication of the likelihood of success for a particular patient or for the diagnosis. In some implementations, the treatment model 126 may also present treatment options in ranked order based on learned preferences of a provider.

The diagnostic and treatment network 125 may be implemented using various data structures, such as graphs or neural networks. Data stored at the diagnostic and treatment network 125 may include data received by and generated by the diagnostic model 124 and the treatment model 126. For example, in some implementations, the diagnostic and treatment network may store diagnostic data, diagnosis, selected treatment, and treatment outcomes in a neural network. The data may be anonymized (e.g., each patient and/or provider may be represented by an anonymizing identifier and any patient identifying information is removed). Various data points may also be tagged with unique identifiers to help aggregate and better analyze data stored at the diagnostic and treatment network 125.

For example, a symptom described by a patient as part of the diagnostic input may be tagged with an identifier describing location, duration, and characteristics of the pain. Such identifiers may allow for comparisons of data points given differences in description and across spoken or written languages.

In some implementations, the diagnostic and treatment network 125 may be routinely updated as new data points are received and generated by the diagnostic model 124 and the treatment model 126. The diagnostic and treatment system 102 may further include various agents configured to interrogate the diagnostic and treatment network 125 to find associations between data points in the diagnostic and treatment network 125. In some embodiments, agents interrogating the diagnostic and treatment network 125 may also update models or algorithms implemented by the diagnostic model 124 and/or the treatment model 126. Such complex adaptive systems may provide associations between large sets of patient and treatment data which would otherwise be unidentified, and may improve upon diagnostic and treatment methods implemented without similar data associations.

Storage 136 may be located locally to or remote from the diagnostic and treatment system 102. For example, in some implementations, the diagnostic and treatment system 102 may be implemented using processors located at a server and storage 136 may be located at the same server. In some implementations, storage 136 may be distributed across multiple physical locations, such as in a cloud services environment or through use of virtual file systems. Storage 136 may be nonvolatile storage including data used by the diagnostic model 124 and the treatment model 126. In some implementations, the patient device 104 and the provider device 106 may directly access storage 136 while, in other implementations, requests from the patient device 104 and the provider device 106 may be handled using the communications interface 128 of the diagnostic and treatment system 102. In some implementations, data and other information on storage 136 may be encrypted, restricted, or otherwise protected from unauthorized access.

Patient data 130 may include, for example, historical information about patient responses to diagnostic questions, selected diagnoses, selected treatments, patient compliance with treatments, patient outcomes, etc. In some implementations, data included in patient data 130 is anonymized, such that particular patients corresponding to data may be unidentifiable except by the patient corresponding to a patient identifier. In some implementations, additional patient data, such as medical history and other medical conditions, may be obtained from other sources, such as a patient's online medical records, and anonymized before being stored with patient data 130. Patient data 130 may be accessed by the diagnostic and treatment system 102 for use in training, refining, or using the diagnostic model 124, the diagnostic and treatment network 125, and the treatment model 126. In some implementations, patient data 130 may also be used for education of providers and students. In some implementations, patient data 130 may be partitioned or sorted by, for example, provider, specialty, practice, or other significant groupings.

Provider data 134 may include data regarding different providers using the diagnostic and treatment system 102. For example, provider data 134 may include statistics on diagnoses treated by a provider, treatments generally used by the provider, a provider's success rates in treating patients with a certain condition, etc. In some implementations, provider data 134 may be anonymized. In other implementations, provider data 134 is not anonymized and may be accessible by patients to assist in choosing a provider to address a particular concern. Provider data 134 may also be used by the treatment model 126 to generate treatment options in accordance with a provider's historical preferences or success rates.

Knowledge center 132 may include, for example, patient education information regarding diagnoses, treatments, providers, etc. In some implementations, the diagnostic and treatment system 102 may automatically send information from the knowledge center 132 to a patient device 104 when a provider selects a diagnosis or treatment for the patient. In some implementations, the provider device 106 may access the knowledge center 132 to choose particular information to send to a patient device 104 or other devices, such as an associated caregiver device.

In various embodiments the diagnostic and treatment system 102, the patient device 104, and the provider device 106 may communicate via a network 108 (shown in FIG. 1). In various embodiments, the network 108 may include the Internet, a local area network (“LAN”), a wide area network (“WAN”), and/or other data network. In addition to traditional data-networking protocols, in some embodiments, data may be communicated according to protocols and/or standards including near field communication (“NFC”), Bluetooth, power-line communication (“PLC”) and the like. In some embodiments, the network 108 may also include a voice network that conveys not only voice communications, but also non-voice data such as Short Message Service (“SMS”) messages, as well as data communicated via various cellular data communication protocols, and the like.

In some implementations, the diagnostic model 124 and the treatment model 126 may be updated as more information is gathered through use of the diagnostic and treatment system 102 to treat patients. For example, an algorithm of the diagnostic model 124 may be adjusted depending on which diagnoses are selected from the subset of differential diagnoses that are presented to a provider. As such, the diagnostic model 124 incorporates and accumulates knowledge from all providers using the diagnostic and treatment system 102 to diagnose and treat patients. In some implementations, the diagnostic and treatment network 125 may be used, analyzed, or interrogated to update the diagnostic model 124 and/or the treatment model 126. For example, agents executing at processors of the diagnostic and treatment system 102 may interrogate the diagnostic and treatment network 125 to evaluate correlations and relationships between various data points. Such correlations may be useful in updating algorithms or models used by the diagnostic model 124 and the treatment model 126. For example, if, when interrogating the diagnostic and treatment network 125, an agent finds that a common medication for treatment of a condition is generally not effective in a subset of patients, the treatment model 126 may be updated to not suggest the medication for the subset of patients. Similar relationships and correlations may be used to update the diagnostic model 124.

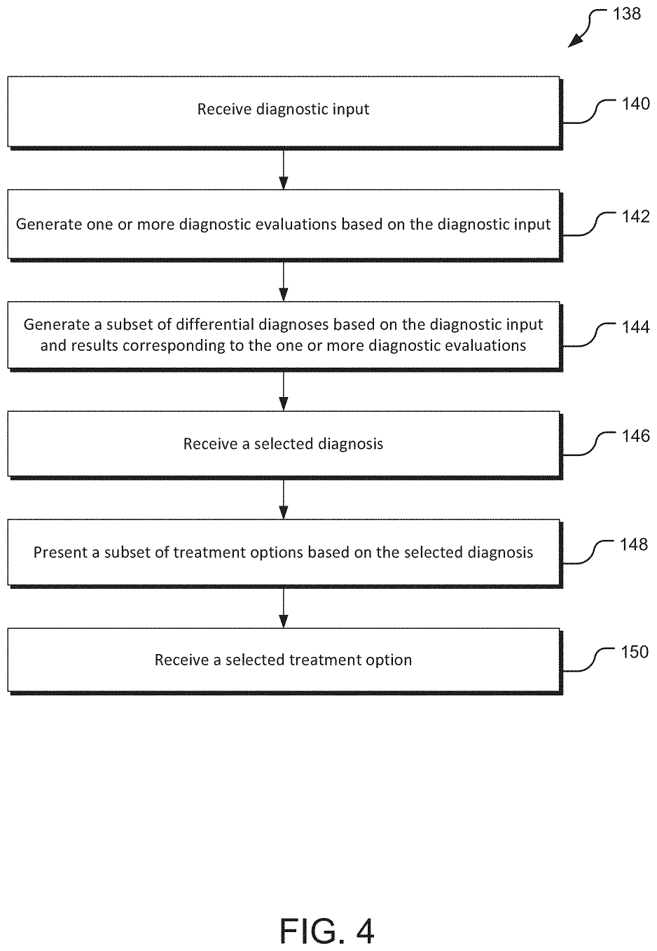

FIG. 4 is a flow diagram for use of an example diagnosis and treatment system. At step 140, the diagnostic and treatment system 102 receives diagnostic input. The diagnostic input may take several forms, including biometric data (e.g., data collected from patient wearable or implanted devices), diagnostic testing results (including text data, image data, etc.), patient answers to diagnostic questions, patient history, images of an affected area, and the like. In various implementations, more than one type of diagnostic input may be provided to the system 102 and the type of diagnostic input provided may vary based on the type of medical problem being treated.

In some implementations, the diagnostic input may be formatted as answers to diagnostic questions initially presented by the diagnostic and treatment system 102. The diagnostic questions may be presented to the patient device 104 or to the provider device 106. For example, the questions may be presented to the patient device 104 to allow the patient to answer the questions and provide information before seeing a provider. In another example, the questions may be presented to the provider device 106 to allow the provider to ask the patient the questions and obtain the patient's history while providing input to the diagnostic and treatment system 102.

The diagnostic questions may be presented, for example, using a display of the patient device 104. The patient may then use an input device (e.g., a touchscreen, mouse, or keyboard) to select an answer to the question on the patient device 104. In other implementations, questions may be presented as an audio, visual, or other output and the patient (or another person, such as a caregiver or provider) may answer the questions using a microphone of the patient device 104. Other input devices of the patient device 104, such as cameras, may be used to provide information to the diagnostic and treatment system 102.

Diagnostic questions may be presented in a variety of formats. For example, some diagnostic questions may be multiple choice (e.g., right eye or left eye). Some multiple choice questions may include an option for “other” where the patient can provide additional information. For example, a question may ask the patient to describe pain by presenting options for sharp, dull, throbbing, or other. A patient may select other and write in “stinging.” Questions may also include options for “not applicable.” Diagnostic questions may also be presented as open-ended questions allowing the patient to type, dictate, or otherwise select an answer. For example, “how long have your symptoms been present?” or “describe the onset of your symptoms” may be presented as open-ended questions. Further, where appropriate, the system may ask for additional types of input, such as an image of the patient's affected eye when the patient lists “eye redness” as a symptom.

In some implementations, additional information may be presented with the diagnostic question. For example, a patient may be asked to choose between several pictures to choose the picture that looks most like the patients affected eye. In other examples, some questions may include rudimentary diagnostic tests, such as vision or hearing screenings. In some implementations, the diagnostic and treatment system 102 may request to access additional information that may be available through the patient's device, such as an electronic medical record, fitness or health tracking data, or other relevant information.

Where diagnostic questions are presented, the diagnostic model 124 of the diagnostic and treatment system 102 may determine the diagnostic questions to present based on previous answers. The diagnostic model 124 may also present diagnostic questions to the patient or provider based on other types of diagnostic input. For example, if a patient answer indicates that the patient is having eye pain, the diagnostic model 124 may select a next question about characteristics of the pain (e.g., sharp, dull, or throbbing). The selection of a next question may be implemented using, for example, a decision tree. For example, using a decision tree, the diagnostic model 124 may eliminate certain further questions based on answers to previous questions using statistical and medical information. For example, the diagnostic model 124 may use an expert system to aggregate statistical and medical information and to determine which diagnostic questions should be presented.

After diagnostic questions are presented by the diagnostic and treatment system 102, the system 102 receives patient answers to the presented diagnostic questions.. For example, the diagnostic and treatment system 102 may receive an answer indicating that a patient is experiencing pain, leading to an additional diagnostic question asking the patient to describe the pain. During this process, the communications interface 128 may receive an answer from the patient device 104 and format the answer for input into the diagnostic model 124. In various implementations, formatting an answer for input into the diagnostic model 124 may include tagging the answer with an encoding of a subset of predetermined encodings. Such encodings may identify relevant parts of the answer and assist the diagnostic model 124 in assessing the answer.

The diagnostic model 124 may complete additional processing and may use the answer differently depending on its format. For example, the diagnostic model 124 may use a natural language processor or other language processing system to extract information from a patient's open ended response. In another example, the diagnostic model 124 may use image processing and comparison to process images received by the diagnostic model 124.

At a step 142, the diagnostic and treatment system 102 generates one or more diagnostic evaluations based on the diagnostic input. The evaluations may be generated using the diagnostic model 124 and may be selected to reduce a list of differential diagnoses, rule out diagnoses, etc. Diagnostic evaluations may include traditional diagnostic tests (e.g., blood draws or imaging) or requests for additional information about the patient from the patient, the provider, and/or other sources. In some examples, the diagnostic evaluations may be presented to the provider device 106. The provider may select one or more tests from the presented list of diagnostic tests. After ordering or otherwise obtaining patient results for the selected diagnostic tests, the provider may provide the results to the diagnostic and treatment system 102.

At a step 144, the diagnostic and treatment system 102 generates a subset of differential diagnoses based on the diagnostic input and results corresponding to the one or more diagnostic evaluations. The subset of differential diagnoses may be generated by the diagnostic model 124 based on information received from the patient device 104 (e.g., patient responses to diagnostic questions). In some implementations, the generation of differential diagnoses may occur in parallel with the receipt of diagnostic input. For example, in such implementations, the diagnostic model 124 may use answers to diagnostic questions to determine a next question to present. The diagnostic model 124 may, in these implementations, include a decision tree. The diagnostic model 124 may move through the decision tree based on information received at step 140. The final subset of differential diagnoses may be generated when the diagnostic model 124 reaches a terminal node of the decision tree through this process.

In other implementations, the step 144 may occur after receiving all diagnostic input at step 140 and after receiving results from the one or more diagnostic evaluations generated at step 142. In these implementations, the diagnostic model 124 may include machine learning or artificial intelligence models generated through either supervised or unsupervised learning. For example, a classifier may be trained using anonymized patient data (e.g., patient data 130) including answers to diagnostic questions and associated diagnoses, treatments, and treatment outcomes. The classifier may then use the answers and other diagnostic input received at step 140 to determine the subset of differential diagnoses. In other examples, the diagnostic model 124 may include or use a neural network (e.g., the diagnostic and treatment network 125) and may use clustering techniques to evaluate the diagnostic input and results corresponding to diagnostic evaluations in comparison to similar information received from other patients to generate the subset of differential diagnoses. In some implementations, the generation of differential diagnoses may include identifying similar patients, e.g., patients who answered diagnostic questions in a similar manner and using diagnoses and treatment outcomes corresponding to the similar patients to generate the differential diagnoses.

The subset of differential diagnoses may be presented to a provider through a provider device 106. In some implementations, the differential diagnoses may be presented with additional information, such as relative probabilities, suggested additional diagnostic tests, etc. The differential diagnoses may be presented using a display or other output (e.g., speakers) of the provider device 106. In some implementations, the system 102 may output a single diagnosis or may output the subset of differential diagnoses with indicators of likelihood, rankings, or other information indicating a relative strength of the diagnosis.

In some implementations, the system 102 may receive further diagnostic input after presentation of the subset of differential diagnoses. For example, the optional first receiving operation may receive test results from a provider or other entity for additional diagnostic tests. In one implementation, the provider may use the provider device 106 to select a test result from a drop-down menu or may use the provider device 106 to input specific values or other information derived from a diagnostic test. Information received during the first receiving operation may be fed back into the diagnostic model 124, such that the diagnostic model produces a more accurate set of differential diagnoses over time.

At step 146, the diagnostic and treatment system 102 receives a selected diagnosis from the subset of differential diagnoses generated at step 144. The provider may select from the subset of differential diagnoses using the provider device 106 and return the selected diagnosis to the diagnostic and treatment system 102. In some implementations, the provider may also elect to send the diagnosis to the patient device 104, to a patient's electronic medical record, or to another designated person (e.g., a caregiver or primary care provider). The diagnostic and treatment system 102 may send patient education information (or links to information) about the received diagnosis to the patient device 104 by retrieving the patient education information from the knowledge center 132.

In some implementations, an additional operation may present the patient with additional questions regarding treatment preferences based on the diagnosis received at step 146. The patient's treatment preferences may be considered by the treatment model 126 to generate treatment options. Additionally, the patient's treatment preferences may be presented to the provider via the provider device 106 so that the provider can take the patient's preferences into account when prescribing treatment. Treatment preferences may include, for example, a desire to avoid surgical intervention if possible, a preference for not using a daily medication, or preference for more or less aggressive treatments. Other questions regarding treatment may also be presented to the patient, such as questions regarding allergies, current medications, and current or past medical conditions. Additional questions regarding treatment and treatment options may be presented via the patient device 104 or may be presented via the provider device 106 to allow to the provider to ask the questions and record the patient's answers.

At step 148, the diagnostic and treatment system 102 presents a subset of treatment options based on the selected diagnosis. The subset of treatment options may be generated by the treatment model 126 based on the selected diagnosis. The treatment model 126 may use the patient's answers to the diagnostic questions, any other information received from the patient (e.g., treatment preferences), or any other information about the patient (e.g., history received from a patient's electronic medical record) to generate the subset of treatment options. In some implementations, the treatment model 126 may include a set subset of treatment options for each diagnosis and the subset may be augmented for a particular patient based on information about the patient. For example, a treatment option may be removed from the set subset of treatment options where the patient has an allergy to the drug used in treatment.

In some implementations, the treatment model 126 may include algorithms, machine learning models, or other models for each possible diagnosis to generate the subset of treatment options. For example, in one implementation, the treatment model 126 may include a trained classifier for each diagnosis, where the classifier is trained using anonymized previous patient data, selected treatments, and treatment outcomes. Any information about the patient may be provided to the classifier in the treatment model 126 to determine treatment options that are most likely to be effective for the patient. In other implementations, unsupervised machine learning models, such as neural networks, may be used similarly within the treatment model 126. In some implementations, the treatment model 126 may output a single treatment option or may output the subset of treatment options with rankings, indicators of likelihood of effectiveness, or other information about the treatment options.

The treatment model 126 may additionally use information about the provider in generating the list of treatment options. For example, provider data 134 may include a provider's historically selected treatment options for a particular diagnosis. The treatment model 126 may access the provider data 134 to present a subset of treatment options in accordance with the provider's preferred treatment. The system 102 may also present additional information generated by the treatment model 126, such as historical success rates for treatment options or predicted chances of a success of the treatment options for the patient. In some implementations, the system 102 may also present a cumulative average of costs per treatment option. These cumulative average costs may be determined by, for example, average cost of a medication by survey or by insurance company, cost of a diagnostic test based on reimbursements, and average number of visits until the condition is managed or treated successfully. The cumulative average costs may be determined using data generated by the diagnostic and treatment system 102, supervised learning, unsupervised learning, publicly available data, or other simulations.

At step 148, the diagnostic and treatment system 102 may present the treatment options to the provider device 106 so that the provider can select treatment from the treatment options. In some implementations, the diagnostic and treatment system 102 may provide the patient with patient education information about a selected treatment option upon receipt of the selected treatment from the provider device 106. Patient education information may be retrieved, for example, from the knowledge center 132.

At step 150, the diagnostic and treatment system 102 receives a selected treatment option. In some implementations, additional operations may include monitoring a patient's treatment compliance. For example, the diagnostic and treatment system 102 may present an interface to the patient via the patient device 104 asking the patient to perform an action (e.g., checking a box) when the patient has performed some aspect of the prescribed treatment (e.g., using prescribed eye drops). The diagnostic and treatment system 102 may save these results as treatment compliance data and may return treatment compliance data to the provider. The treatment compliance data may also be stored in an anonymized manner with patient data 130 for later use by the diagnostic model 124 and the treatment model 126.

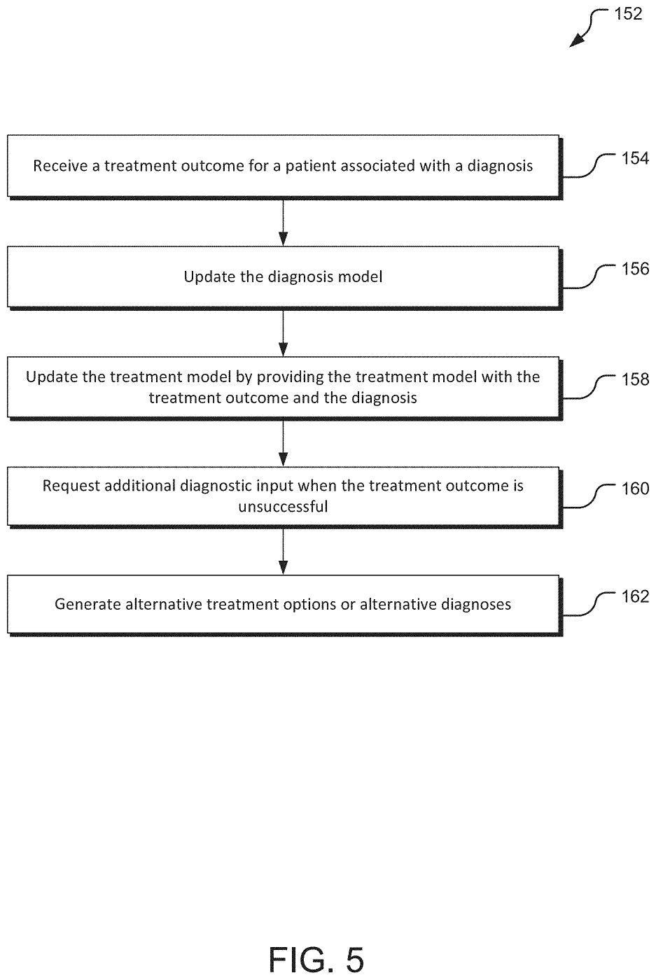

FIG. 5 is flow diagram for use of an example diagnosis and treatment system 102. At step 154, the diagnostic and treatment system 102 receives a treatment outcome for a patient associated with a diagnosis. The treatment outcome may be received, for example, from the patient device 104 responsive to questions about the treatment outcome presented to the patient device 104. For example, in some implementations, the diagnostic and treatment system 102 may present additional questions to the patient once the chosen treatment is completed. In some implementations, the treatment outcome may be received from the provider device 106 as part of a follow-up with the patient regarding treatment progress. The diagnostic and treatment system 102 may receive either a “successful” or “unsuccessful” indicator. In some implementations, an “unsure” indicator may also be received. Other indicators of treatment success, such as an alternate diagnosis or additional prescription for another treatment option, may be received at step 154 and interpreted by the diagnostic and treatment system 102 as a successful or unsuccessful treatment outcome.

At step 156, the system 102 updates the diagnosis model 124. The diagnosis model 124 may be updated using the patient's original answers to the diagnostic questions and the treatment outcome. For example, a successful treatment outcome may indicate that the selected diagnosis is correct such that future patients providing the same answers to diagnostic questions may be more likely to receive the diagnosis. In another example, when the received treatment outcome is unsuccessful, future patients providing the same answers to diagnostic questions may be less likely to receive the diagnosis.

The updating may occur automatically, with additional input from the provider via the provider device 106, or with additional input from another person, such as an administrator. For example, a provider may suggest or implement additional diagnostic questions that, if asked, would have presented a more accurate list of differential diagnoses. In other examples, the diagnostic model 124 may be provided with data as a labeled observation such that the diagnostic model 124 learns and becomes more accurate over time. In some implementations, updating may be implemented using an expert system including an inference engine updating a knowledge base of the diagnostic model 124.

At step 158, the system 102 updates the treatment model 126. The treatment model 126 may be updated by providing the treatment model 126 with the treatment outcome and the diagnosis. For example, a successful treatment outcome paired with a diagnosis may update the treatment model 126 such that it is more likely that the selected treatment option will be presented for the diagnosis in the future. Similarly, an unsuccessful treatment outcome may update the treatment model 126 such that it is less likely that the selected treatment option will be presented for the diagnosis in the future. The treatment model 126 may also use additional information to update such as patient treatment compliance information or other historical information about the patient collected by the diagnostic and treatment system 102. The treatment model 126 may be updated automatically or with additional input from a provider or administrator. The diagnostic and treatment system 102 may also include additional information about the patient, such as underlying conditions or physical issues, to update the treatment model 126. Such information may help to determine why treatments are effective for some patients and ineffective for others. In some implementations, the treatment model 126 may be updated by an inference engine updating a knowledge base of the treatment model 126. Updating creates an adaptive treatment model 126 which becomes more accurate and sophisticated over time.

In some implementations, an additional operation may anonymize the patient data, including some or all of the patient's answers to diagnostic questions, diagnosis, treatment, treatment outcome, and treatment compliance data. The anonymized data may then be stored with other patient data 130 for use by the diagnostic and treatment system 102. The anonymized patient data, along with an identifier of the provider, may also be stored with provider data 134 for use by the diagnostic and treatment system 102.

At step 160, the diagnostic and treatment system 102 requests additional diagnostic input when the treatment outcome is unsuccessful. In some examples, the system 102 may present additional diagnostic questions when the treatment outcome is unsuccessful. The additional diagnostic questions may be presented to the provider device 106, the patient device 104, or another device. The additional diagnostic questions may be selected by either or both of the diagnostic model 124 and the treatment model 126 to assist the provider in determining whether to select a new diagnosis and treatment option or to keep the diagnosis and try a different treatment option.

At step 162, the diagnostic and treatment system 102 generates alternative treatment options or alternative diagnoses. The alternative treatment options or alternative diagnoses may be generated based on the additional diagnostic question presented at step 160. Additional statistics, such as predicted success rate and historical success rates of treatments may be presented with alternative treatment options or alternative diagnoses. The diagnostic and treatment system 102 may generate the alternative treatment options and/or alternative diagnoses using the diagnosis model 124 and/or the treatment model 126 as described herein.

In some implementations, after selecting an updated diagnosis or treatment option, the provider or the diagnostic and treatment system 102 may send additional patient education information from the knowledge center 132 to the patient device 104.

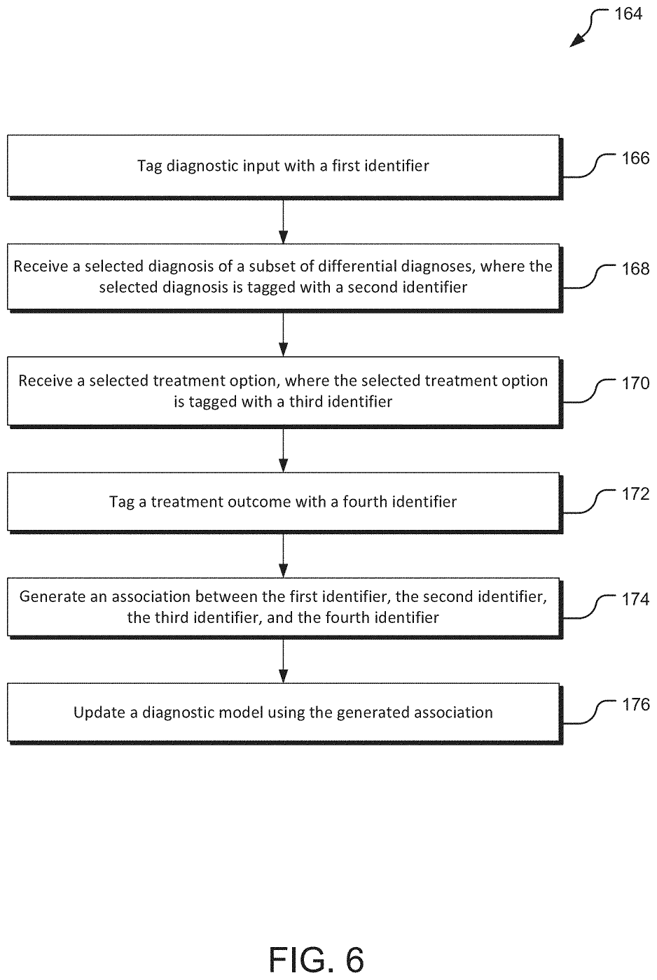

FIG. 6 is a flow diagram of steps using an example diagnosis and treatment system 102. The steps shown in FIG. 6 may allow the system to tag and/or encode various types of data entered into the system at various decision points. The tagged or encoded data may be used both to build a meaningful dataset (e.g., at the diagnostic and treatment network 125), as well as to enhance and increase accuracy of the system's output by providing feedback to various models of the system (e.g., the diagnostic model 124 and the treatment model 126).

At step 166, the system 102 tags diagnostic input with a first identifier. The identifier may be from a subset of pre-defined identifiers, may be dynamically generated, etc.. The pre-defined identifiers may encode relevant information from the diagnostic input to allow meaningful analysis of data and translation between spoken languages. For example, identifiers may be an alphanumeric or other code including information such as input type, affected area of the body, symptoms, characteristics of symptoms, objective findings, and the like. For example, an identifier may encode that diagnostic input was received as a patient response to a diagnostic question, and that the patient has experienced burning in the right eye for a duration of approximately two weeks. Accordingly, the identifier is generally not dependent on how the patient phrases their answers or which spoken language is used.

At step 168, the system 102 receives a selected diagnosis of a subset of differential diagnoses, where the selected diagnosis is tagged with a second identifier. The second identifier may be generated using the same or a similar system as the first identifier. The second identifier may encode information such as the ICD10 code of a diagnosis, location in the body of the diagnosis, stage of the diagnosis, sub-description or sub-type of the diagnosis, and the like.

At step 170, the diagnostic and treatment system 102 receives a selected treatment option, where the selected treatment option is tagged with a third identifier. The identifier for the selected treatment option may be encoded or generated using the same or a similar system as the first identifier. The third identifier may encode information such as type of treatment (e.g., pharmaceutical, surgical, other intervention), frequency, dosages, manufacturers, class of pharmaceutical, type of procedure, and the like. In some implementations, a treatment may include several options or treatment modalities in combination. Accordingly, the third identifier may reflect the combination of treatments and/or additional identifiers may be used to reflect the combination of treatments.

At step 172, the system 102 tags a treatment outcome with a fourth identifier. The identifier for the treatment outcome may be encoded or generated using the same or a similar system as the first identifier. Various information may be encoded by the fourth identifier including, for example, degree of improvement (e.g., partial symptom response, total symptom response, or no response), objective response (e.g., based on changes in diagnostic tests), timeframe of treatment outcome, recurrence of symptoms, patient compliance with treatment, and the like.

At step 174, the system 102 generates an association between the first identifier, the second identifier, the third identifier, and the fourth identifier. The association may, in some implementations, combine the identifiers as a data point, such that the course of diagnosis and treatment for the patient is stored by the combination of the identifiers.

At step 176, the diagnostic and treatment system 102 updates the diagnostic model 124 using the generated association. In some implementations, the association between the first, identifier, the second identifier, the third identifier, and the fourth identifier may be stored in a knowledge base of the diagnostic and treatment system 102, such as the diagnostic and treatment network 125. In these implementations, the updates to the diagnostic and treatment network 125 may be utilized to update the diagnostic model 124 and/or the treatment model 126. For example, the association between the identifiers may be stored in the diagnostic and treatment network 125, updating the diagnostic and treatment network 125. Agents or other software entities interrogating and/or analyzing the diagnostic and treatment network 125 may uncover new or different patterns, correlations, or other information in the network when the association is added. The new information obtained by the agents from the network 125 may be used to update, change, streamline, or otherwise alter models, algorithms, decision trees, or other structures implemented within the diagnostic model 124 and/or the treatment model 126.

The technology described herein may be implemented as logical operations and/or modules in one or more systems. The logical operations may be implemented as a sequence of processor-implemented steps directed by software programs executing in one or more computer systems and as interconnected machine or circuit modules within one or more computer systems, or as a combination of both. Likewise, the descriptions of various component modules may be provided in terms of operations executed or effected by the modules. The resulting implementation is a matter of choice, dependent on the performance requirements of the underlying system implementing the described technology. Accordingly, the logical operations making up the embodiments of the technology described herein are referred to variously as operations, steps, objects, or modules. Furthermore, it should be understood that logical operations may be performed in any order, unless explicitly claimed otherwise or a specific order is inherently necessitated by the claim language.

In some implementations, articles of manufacture are provided as computer program products that cause the instantiation of operations on a computer system to implement the procedural operations. One implementation of a computer program product provides a non-transitory computer program storage medium readable by a computer system and encoding a computer program. It should further be understood that the described technology may be employed in special purpose devices independent of a personal computer.

The above specification, examples and data provide a complete description of the structure and use of exemplary embodiments of the invention as defined in the claims. Although various embodiments of the claimed invention have been described above with a certain degree of particularity, or with reference to one or more individual embodiments, it is appreciated that numerous alterations to the disclosed embodiments without departing from the spirit or scope of the claimed invention may be possible. Other embodiments are therefore contemplated. It is intended that all matter contained in the above description and shown in the accompanying drawings shall be interpreted as illustrative only of particular embodiments and not limiting. Changes in detail or structure may be made without departing from the basic elements of the invention as defined in the following claims.

1. A method comprising:
communicating with a user device to obtain diagnostic input;
generating one or more diagnostic evaluations based on the diagnostic input;
generating by a model a subset of differential diagnoses based on the diagnostic input and results corresponding to the one or more diagnostic evaluations, wherein the diagnostic input and the results corresponding to the one or more diagnostic evaluations;
receiving a selected diagnosis from the subset of differential diagnoses;
generating a subset of treatment options based on the selected diagnosis; and
receiving a selected treatment of the subset of treatment options; and
updating the model using a treatment outcome for the selected treatment option of the subset of treatment options.

2. The method of claim 1, wherein the treatment outcome includes a representation of patient compliance with the selected treatment option.

3. The method of claim 1, further comprising:
displaying the subset of treatment options, wherein one or more of the displayed treatment options includes a measure of treatment success.

4. The method of claim 3, wherein the measure of treatment success is determined based on stored data regarding treatment outcomes for patients with a same diagnosis.

5. The method of claim 1, wherein the diagnostic input, the selected diagnosis, and the selected treatment are tagged with an identifier from a subset of pre-defined identifiers.

6. The method of claim 1, further comprising:
generating the treatment outcome based on a measure of compliance with the selected treatment option and on an adaptation of knowledge accumulated in the model.

7. The method of claim 6, wherein the measure of compliance with the selected treatment option is generated based on compliance data collected through the user device.

8. The method of claim 6, wherein updating the model comprises updating the model using the treatment outcome, selected diagnosis, and the measure of compliance with the selected treatment option.

9. The method of claim 1, wherein the model comprises a classifier or expert system generated using seeded data.

10. A system comprising:
a communications interface configured to communicate with a user device to receive diagnostic input from the user device;
a diagnostic model configured to generate a subset of differential diagnoses based on the diagnostic input; and
a treatment model configured to generate a subset of treatment options based on a selected diagnosis selected from the subset of differential diagnoses;
wherein the diagnostic model and the treatment model are configured to update based on a treatment outcome for a selected treatment option from the subset of treatment options.

11. The system of claim 10, further comprising:
a database configured to communicate with the diagnostic model and the treatment model, wherein the database includes anonymized patient data for a plurality of patients previously evaluated using the system.

12. The system of claim 11, further comprising:
a neural network generated based on the database, wherein the diagnostic model and the treatment model are updated based on patterns obtained by interrogation of the neural network.

13. The system of claim 12, wherein the anonymized patient data is further tagged with an identifier selected from a subset of pre-defined identifiers.

14. The system of claim 10, further comprising:
a knowledge database including patient education information, wherein the communications interface is further configured to communicate with the patient device to communicate selected patient education information from the knowledge database, the selected patient education information being selected based on the answers to the diagnostic questions.

15. The system of claim 10, wherein the diagnostic model comprises a classifier or expert system generated using seeded data.

16. The system of claim 15, wherein the diagnostic model further comprises an image analysis model.

17. At least one non-transitory computer-readable media encoded with instructions for implementing a system, the instructions comprising instructions for:
communicating with a patient device to obtain diagnostic input;
generating one or more diagnostic evaluations based on the diagnostic input;
generating by a diagnostic model a subset of differential diagnoses based on the diagnostic input and results corresponding to the one or more diagnostic evaluations, wherein the diagnostic input and the results corresponding to the one or more diagnostic evaluations;
receiving a selected diagnosis from the subset of differential diagnoses; and
generating a subset of treatment options based on the selected diagnoses.

18. The at least one non-transitory computer-readable media of claim 17, wherein the instructions further comprise instructions for:
generating a neural network based on data for a plurality of patients previously evaluated using the model; and
updating the model based on patterns obtained by interrogation of the neural network.

19. The at least one non-transitory computer-readable media of claim 17, wherein the model comprises a classifier or expert system generated using seeded data.

20. The at least one non-transitory computer-readable media of claim 17, wherein the diagnostic input, the selected diagnosis, and the selected treatment are tagged with an identifier from a subset of pre-defined identifiers.
